(19) United States
(12) Patent Application Publication
(10) Pub. No.: US 2006/0211648 A1
(43) Pub. Date: Sep. 21, 2006

(54) Naphthalimide compositions and uses thereof

(75) Inventor: Dennis M. Brown, Menlo Park, CA (US)

Correspondence Address: Richard F. Trecartin;Dorsey & Whitney LLP, Intellectual Property Department, 555 California Street, Suite 1000, San Francisco, CA, 94104-1513, US

(73) Assignee: ChemGenex Pharmaceuticals, Inc.

(21) Appl. No.: 11/356,916

(22) Filed: Feb. 16, 2006

Related U.S. Application Data
(63) Continuation-in-part of application No. 11/067,074, filed on Feb. 25, 2005.
Application No. 11/067,074 is a continuation of application No. 10/273,801, filed on Oct. 17, 2002, now abandoned.
(63) Continuation-in-part of application No. 09/834,177, filed on Apr. 12, 2001, now Pat. No. 6,630,173.
(60) Provisional application No. 60/330,037, filed on Oct. 17, 2001.
(60) Provisional application No. 60/197,103, filed on Apr. 12, 2000.

(51) Int. Cl. A61K 31/7072; A61K 31/473

(52) U.S. Cl. 514/50; 514/296

(57) ABSTRACT

A method of treatment of a host with a cellular proliferative disease, comprising contacting the host with a naphthalimide and an antiproliferative agent, each in an amount sufficient to modulate said cellular proliferative disease, is described. In some embodiments, the naphthalimide comprises amonafide (5-amino-2-[2-(dimethylamine)ethyl]-1H-benz[de-]isoquinoline-1,3-(2H)-dione). Antiproliferative agents of the invention comprise alkylating agents, intercalating agents, metal coordination complexes, pyrimidine nucleosides, purine nucleosides, inhibitors of nucleic acid associated enzymes and proteins, and agents affecting structural proteins and cytoplasmic enzymes. The invention comprises the described methods as well as compositions and kits comprising a naphthalimide and an antiproliferative agent.

### CROSS-REFERENCE TO RELATED APPLICATIONS

This is a continuation-in-part of U.S. Ser. No. 11/067,074 filed Feb. 25, 2005, which is a continuation of U.S. Ser. No. 10/273,801 filed Oct. 17, 2002, and claims the benefit of U.S. Provisional Application No. 60/330,037, filed Oct. 17, 2001, and is a continuation-in-part application of U.S. Ser. No. 09/834,177, filed Apr. 12, 2001, which claims the benefit of U.S. Provisional Application No. 60/197,103, filed Apr. 12, 2000, all of which are hereby incorporated by reference in their entirety.

### FIELD OF THE INVENTION

The technical field of the invention is the use of naphthalimides with antiproliferative agents to treat a host with a cellular proliferative disease.

### BACKGROUND OF THE INVENTION

There is considerable interest in modulating the efficacy of currently used antiproliferative agents to increase the rates and duration of antitumor effects associated with conventional antineoplastic agents.

Conventional antiproliferative agents used in the treatment of cancer are broadly grouped as (1) chemical compounds which affect the integrity of nucleic acid polymers by binding, alkylating, inducing strand breaks, intercalating between base pairs or affecting enzymes which maintain the integrity and function of DNA and RNA; (2) chemical agents that bind to proteins to inhibit enzymatic action (e.g., antimetabolites) or the function of structural proteins necessary for cellular integrity (e.g., antitubulin agents). Other chemical compounds that have been identified to be useful in the treatment of some cancers include drugs which block steroid hormone action for the treatment of breast and prostate cancer, photochemically activated agents, radiation sensitizers, and protectors.

Of special interest to this invention are those compounds that directly affect the integrity of the genetic structure of the cancer cells. Nucleic acid polymers such as DNA and RNA are prime targets for anticancer drugs. Alkylating agents such as nitrogen mustards, nitrosoureas, aziridine containing compounds directly attack DNA. Metal coordination compounds such as cisplatin and carboplatin similarly directly attack the nucleic acid structure resulting in lesions that are difficult for the cells to repair which, in turn, can result in cell death. Other nucleic acid affecting compounds include anthracycline molecules such as doxorubicin, which intercalates between the nucleic acid base pairs of DNA polymers, bleomycin, which causes nucleic acid strand breaks, fraudulent nucleosides such as pyrimidine and purine nucleoside analogs, which are inappropriately incorporated into nucleic polymer structures and ultimately cause premature DNA chain termination. Certain enzymes that affect the integrity and functionality of the genome can also be inhibited in cancer cells by specific chemical agents and result in cancer cell death. These include enzymes that affect ribonucleotide reductase (e.g., hydroxyurea, gemcitabine), topoisomerase I (e.g., camptothecin) and topoisomerase II (e.g., etoposide).

One of the most broadly used of these DNA targeted anticancer drugs is cisplatin (cis-diamminedichloroplatinum II, CDDP). This compound is active against several human cancers including testicular, small-cell lung, bladder, cervical and head and neck cancer.

Although the clinical activity of currently approved antiproliferative agents against many forms of cancers can be shown, improvements in tumor response rates, duration of response and ultimately patient survival are still sought. The invention described herein demonstrates the novel use of the naphthalimides and analogs thereof, including amonafide, which can potentiate the antitumor effects of chemotherapeutic drugs, in particular, agents affecting the integrity of nucleic polymers such as DNA.

### SUMMARY OF THE INVENTION

Methods and compositions are provided for the treatment of a host having a cellular proliferative disease, particularly a neoplasia. In the subject methods, pharmaceutically acceptable naphthalimide and cytarabine are administered in an amount sufficient to modulate the cellular proliferative disease.

### DETAILED DESCRIPTION OF THE FIGURES

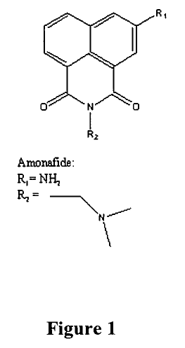

FIG. 1 depicts the general structure of a naphthalimide analog. R₁ and R₂ represent substitution groups. The structures of R₁ and R₂ for the naphthalimide analog, amonafide, are shown.

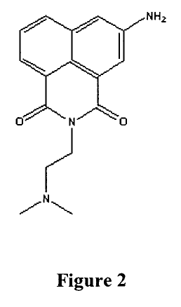

FIG. 2 depicts the structure of the naphthalimide analog, amonafide.

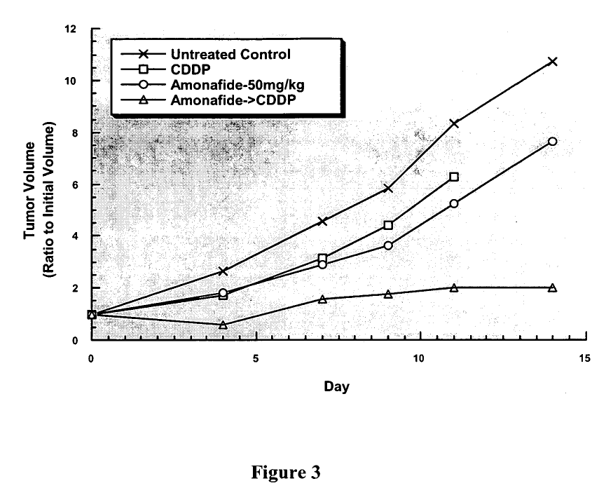

FIG. 3 shows tumor growth delay, as tumor volume on days after treatment with the naphthalimide analog, amonafide, amonafide followed by CDDP, or CDDP alone.

### DETAILED DESCRIPTION OF THE INVENTION

Methods and compositions are provided for the treatment of a host with a cellular proliferative disease, particularly a neoplasia. In the subject methods, a pharmaceutically acceptable naphthalimide is administered, preferably systemically, in conjunction with an antiproliferative agent to improve the anticancer effects. In a preferred embodiment, the naphthalimide provides a chemopotentiator effect.

Methods and compositions are provided herein for the treatment of a host. A “host” for the purposes of the present invention includes both humans and other animals, particularly mammals, and organisms. Thus the methods are applicable to both human therapy and veterinary applications. In the preferred embodiment the patient is a mammal, and in the most preferred embodiment the patient is human.

The methods of the invention are used to treat a cellular proliferative disease. According to a preferred embodiment, the cellular proliferative disease is a tumor, e.g., a solid tumor. Solid tumors that are particularly amenable to treatment by the claimed methods include carcinomas and sarcomas. Carcinomas include those malignant neoplasms derived from epithelial cells which tend to infiltrate (invade) the surrounding tissues and give rise to metastases. Adenocarcinomas are carcinomas derived from glandular tissue or in which the tumor cells form recognizable glandular structures. Sarcomas broadly include tumors whose cells are embedded in a fibrillar or homogeneous substance like embryonic connective tissue.

It will be understood that the method of the invention is not limited to the treatment of these tumor types, but extends to any solid tumor derived from any organ system.

Cellular proliferative diseases that can be treated by the methods and compositions of the invention include, for example, psoriasis, skin cancer, viral induced hyperproliferative HPV-papiloma, HSV-shingles, colon cancer, bladder cancer, breast cancer, melanoma, ovarian carcinoma, prostatic carcinoma, or lung cancer, and a variety of other cancers as well.

The agents are provided in amounts sufficient to modulate a cellular proliferative disease. In one embodiment, modulation of a cellular proliferative disease comprises a reduction in tumor growth. In another embodiment, modulation of a disease comprises inhibition of tumor growth. In another embodiment, modulation of a cellular proliferative disease comprises an increase in tumor volume quadrupling time (described below). In another embodiment, modulation of a cellular proliferative disease comprises a chemopotentiator effect. In another embodiment, modulation of a disease comprises a chemosensitizing effect. In other embodiments, modulation of a disease comprises cytostasis. In still other embodiments, modulation of a disease comprises a cytotoxic effect.

The agents are administered to a host by a variety of routes. According to one embodiment, a naphthalimide is administered by injection, preferably by parenteral, e.g., intravenous, injection. According to one embodiment, an antiproliferative agent is administered by injection, preferably by intravenous injection. The mode of administration of the agents may be the same or different for each. Thus, the compounds may be administered in a single dosage form, one may be administered orally and the other intravenously, one may be administered continuously and the other intermittantly, etc.

Any suitable route of administration may be employed for providing a mammal, especially a human, with an effective dosage of the compounds of the invention. For example, oral, rectal, topical, parenteral, ocular, pulmonary, nasal, etc. routes may be employed. Dosage forms include tablets, troches, dispersions, suspensions, solutions, capsules, creams, ointments, aerosols, and the like. The routes of administration may be the same or different for each of the two compounds.

Disclosed herein are methods of treatment comprising contacting a host with a naphthalimide in conjunction with an antiproliferative agent. By “in conjunction with” is meant that the two agents are administered such that both agents are present and active in the host together during at least a portion of the treatment schedule. According to one embodiment, the two agents are administered simultaneously, in a single dosage form.

According to an alternative embodiment, the administration of one agent is followed by administration of the other agent. For example, administration of a naphthalimide may be followed by administration of an antiproliferative agent; or administration of an antiproliferative agent may be followed by administration of a naphthalimide.

When administration of the two agents is not simultaneous, a defined length of time may separate the two agents. According to one embodiment, administration of each agent is separated by at least about 5 minutes but by no more than 4 hours. Generally, when administration of the two agents is not simultaneous, the time separating the administration of each agent is no more than two plasma half lives of the first administered agent. According to a preferred embodiment, administration of each agent is separated by about 30 minutes. According to another embodiment, administration of each agent is separated by about 1 hour. According to another embodiment, administration of each agent is separated by about 2 hours.

The optimal time separating the administration of the agents will vary depending on the dosage used, the clearance rate of each agent, and the particular host treated. According to the claimed methods, the naphthalimide and the antiproliferative agent used are administered such that the agents are both present together in the host system in active form during the treatment of the host. That is, the agent that is administered first will be present in the host in an active form after the second agent is administered.

A chemical agent is a “chemopotentiator” when it enhances the effect of a known antiproliferative drug in a more than additive fashion relative to the activity of the chemopotentiator or antiproliferative agent used alone. In some cases, a “chemosensitizing” effect may be observed. This is defined as the effect of use of an agent that if used alone would not demonstrate significant antitumor effects but would improve the antitumor effects of an antiproliferative agent in a more than additive fashion than the use of the antiproliferative agent by itself.

As used herein, the term “naphthalimide” includes all members of that chemical family including benzisoquinolinedione and analogs thereof. The naphthalimide family is defined by chemical structure as depicted in FIG. 1.

A naphthalimide analog is further defined but not limited to substituent changes in R₁ and R₂ (FIG. 1). Examples of R₁ and R₂ include those listed in Table 1. In a preferred embodiment, a naphthalimide analog has the structure of amonafide, shown in FIG. 2.

TABLE 1

| Group | Substitution | Length |
|---|---|---|
| R₁ | Alkyl | C₁ → C₅ |
| | Amino | |
| | Nitro | |
| | Cyano | |
| | Alkoxy | OC₁ → OC₅ |
| | Hydrogen | |
| R₂ | Alkyl | C₁ → C₅ |

“Alkyl” means a straight-chain or branched-chain alkyl radical containing from 1 to 10, preferably from 1 to 6, and more preferably from 1 to 4, carbon atoms. Examples of such radicals include methyl, ethyl, n-propyl, isopropyl, n-butyl, isobutyl, sec-butyl, tert-butyl, pentyl, iso-amyl, hexyl, and decyl. “Alkoxy” means an alkyl ether radical. Examples of alkyl ether radicals include methoxy, ethoxy, n-propoxy, iso-propoxy, n-butoxy, iso-butoxy, sec-butoxy, and tert-butoxy.

A naphthalimide analog is a further chemical refinement. A specific example of a naphthalimide analog is amonafide which is also known by the following chemical synonyms: Nafidamide; Benzisoquinolinedione; 5-amino-2-[(dimethylamine)ethyl]-1H-benz[de-]isoquinoline-1,3-(2H)-dione (FIG. 2).

As used herein, antiproliferative agents are compounds which induce cytostasis or cytotoxicity. “Cytostasis” is the inhibition of cells from growing while “cytotoxicity” is defined as the killing of cells.

Specific examples of antiproliferative agents include: antimetabolites, such as methotrexate, 5-fluorouracil, gemcitabine, cytarabine, pentostatin, 6-mercaptopurine, 6-thioguanine, L-asparaginase, hydroxyurea, N-phosphonoacetyl-L-aspartate (PALA), fludarabine, 2-chlorodeoxyadenosine, and floxuridine; structural protein agents, such as the vinca alkaloids, including vinblastine, vincristine, vindesine, vinorelbine, paclitaxel, and colchicine; agents that affect NF-κB, such as curcumin and parthenolide; agents that affect protein synthesis, such as homoharringtonine; antibiotics, such as dactinomycin, daunorubicin, doxorubicin, idarubicin, bleomycins, plicamycin, and mitomycin; hormone antagonists, such as tamoxifen and luteinizing hormone releasing hormone (LHRH) analogs; nucleic acid damaging agents such as the alkylating agents mechlorethamine, cyclophosphamide, ifosfamide, chlorambucil, dacarbazine, methylnitrosourea, semustine (methyl-CCNU), chlorozotocin, busulfan, procarbazine, melphalan, carmustine (BCNU), lomustine (CCNU), and thiotepa, the intercalating agents doxorubicin, dactinomycin, daurorubicin and mitoxantrone, the topoisomerase inhibitors etoposide, camptothecin and teniposide, and the metal coordination complexes cisplatin and carboplatin.

Cytarabine (Ara-C, cytosine arabinose, and 1-(β-D-arabinofuranosyl)cytosine) is a pyrimidine nucleotide analog having a structure similar to the nucleotide cytosine but having an arabinose sugar instead of a ribose or deoxyribose sugar. The arabinose sugar competes with enzymes involved in DNA synthesis thereby allowing cytarabine to inhibit DNA synthesis. As a result cytarabine can block the transition of cells from the G-phase to the S-phase.

Also claimed herein are pharmaceutical compositions comprising a naphthalimide and an antiproliferative agent. The naphthalimide and antiproliferative agent may be in intimate admixture or they may isolated from each other. According to one embodiment, the claimed pharmaceutical compositions comprise pharmaceutically acceptable salts of a naphthalimide or antiproliferative agent. According to one embodiment, the claimed pharmaceutical compositions may contain pharmaceutically acceptable carriers and, optionally, other therapeutically active ingredients.

In one embodiment, the methods of the invention to treat a cellular proliferative disease involve the use of a composition including a napthalimide and an antimetabolite. In a preferred embodiment, the antimetabolite is cytarabine.

In one aspect, the naphthalamide is administered by the methods described herein at a dosage of about 0.5 mg/kg to about 125 mg/kg, about 1 mg/kg to about 120 mg/kg, about 2 mg/kg to about 115 mg/kg, about 5 mg/kg to about 110 mg/kg, about 10 mg/kg to about 105 mg/kg, about 15 mg/kg to about 100 mg/kg, about 20 mg/kg to about 100 mg/kg, about 25 mg/kg to about 95 mg/kg, about 30 mg/kg to about 90 mg/kg, about 35 mg/kg to about 85 mg/kg, about 40 mg/kg to about 80 mg/kg, about 45 mg/kg to about 75 mg/kg, about 50 mg/kg to about 70 mg/kg, about 55 mg/kg to about 65 mg/kg, about 57 mg/kg to about 63 mg/kg, and preferably about 60 mg/kg.

In another aspect, the cytarabine is administered by the methods described herein at a dosage of between about 1 mg/kg to about 800 mg/kg, about 25 mg/kg to about 775 mg/kg, about 50 mg/kg to about 750 mg/kg, about 75 mg/kg to about 725 mg/kg, about 100 mg/kg to about 700 mg/kg, about 125 mg/kg to about 675 mg/kg, about 150 mg/kg to about 650 mg/kg, about 175 mg/kg to about 625 mg/kg, about 200 mg/kg to about 600 mg/kg, about 225 mg/kg to about 575 mg/kg, about 250 mg/kg to about 550 mg/kg, about 275 mg/kg to about 525 mg/kg, about 300 mg/kg to about 500 mg/kg, about 325 mg/kg to about 475 mg/kg, about 350 mg/kg to about 450 mg/kg, about 375 mg/kg to about 425 mg/kg, and preferably about 400 mg/kg. In another preferred embodiment, the naphthalimide is amonafide. In a most preferred embodiment, the antimetabolite is cytarabine and the naphthalimide is amonafide.

The agents may be provided in a range of concentrations, depending on the cellular proliferative disease to be treated, host species, clearance rate of each agent, drug absorption, bioavailability, mode of administration. In a preferred embodiment, a naphthalimide is provided for administration at between about 1-30 mg/kg or 50-1000 mg/m² In a preferred embodiment, an antiproliferative agent is provided for administration at between about 0.1-50 mg/kg. Generally the concentration administered will depend on a variety of factors, including the dose and schedule that are optimal for the antiproliferative agent used, as known and understood by those of skill in the art.

The compositions include compositions suitable for oral, rectal, topical (including transdermal devices, aerosols, creams, ointments, lotions, and dusting powders), parenteral (including subcutaneous, intramuscular, and intravenous), ocular (ophthalmic), pulmonary (nasal or buccal inhalation), or nasal administration; although the most suitable route in any given case will depend largely on the nature and severity of the condition being treated and on the nature of the active ingredient. The agents may be conveniently presented in unit dosage form and prepared by any of the methods well known in the art of pharmacy.

For example, compounds of the invention may be administered orally, for example in tablet form, or by inhalation, for example in aerosol or other atomisable formulations or in dry powder formulations, using an appropriate inhalation device such as those known in the art. The compounds of the invention may also be administered intranasally.

In the case of oral delivery, the dosage form would allow that suitable concentrations of a naphthalimide would be provided in a form such that an adequate plasma level could be achieved to provide the chemopotentiation of the other chemotherapeutic compound(s). Tablets, capsules, suspensions or solutions may contain 10 milligrams to 2 grams per dose treatment to achieve the appropriate plasma concentrations.

A compound of the invention may be combined as the active ingredient in intimate admixture with a pharmaceutical carrier according to conventional pharmaceutical compounding techniques. The carrier may take a wide variety of forms depending on the nature of the preparation desired for administration, i.e., oral, parenteral, etc. In preparing oral dosage forms, any of the usual pharmaceutical media may be used, such as water, glycols, oils, alcohols, flavoring agents, preservatives, coloring agents, and the like in the case of oral liquid preparations (e.g., suspensions, elixirs, and solutions); or carriers such as starches, sugars, microcrystalline cellulose, diluents, granulating agents, lubricants, binders, disintegrating agents, etc. in the case of oral solid preparations such as powders, capsules, and tablets. Solid oral preparations are preferred over liquid oral preparations. Because of their ease of administration, tablets and capsules are the preferred oral dosage unit form. If desired, capsules may be coated by standard aqueous or non-aqueous techniques.

In addition to the dosage forms described above, the compounds of the invention may be administered by controlled release means and devices.

Pharmaceutical compositions of the present invention suitable for oral administration may be prepared as discrete units such as capsules, cachets, or tablets each containing a predetermined amount of the active ingredient in powder or granular form or as a solution or suspension in an aqueous or nonaqueous liquid or in an oil-in-water or water-in-oil emulsion. Such compositions may be prepared by any of the methods known in the art of pharmacy. In general, the compositions are prepared by uniformly and intimately admixing the active ingredient with liquid carriers, finely divided solid carriers, or both and then, if necessary, shaping the product into the desired form. For example, a tablet may be prepared by compression or molding, optionally with one or more accessory ingredients. Compressed tablets may be prepared by compressing in a suitable machine the active ingredient in a free-flowing form such as powder or granule optionally mixed with a binder, lubricant, inert diluent, or surface active or dispersing agent. Molded tablets may be made by molding in a suitable machine, a mixture of the powdered compound moistened with an inert liquid diluent.

Ophthalmic inserts are made from compression molded films which are prepared on a Carver Press by subjecting the powdered mixture of active ingredient and HPC to a compression force of 12,000 lb. (gauge) at 149° C. for 1-4 min. The film is cooled under pressure by having cold water circulate in the platen. The inserts are then individually cut from the film with a rod-shaped punch. Each insert is placed in a vial, which is then placed in a humidity cabinet (88% relative humidity at 30° C.) for 2-4 days. After removal from the cabinet, the vials are capped and then autoclaved at 121° C. for 0.5 hr.

The inhalable form may be, for example, an atomisable composition such as an aerosol comprising the compounds of the invention in solution or dispersion in a propellant or a nebulizable composition comprising a dispersion of the compound of the invention in an aqueous, organic or aqueous/organic medium, or a finely divided particulate form comprising the compounds of the invention in finely divided form optionally together with a pharmaceutically acceptable carrier in finely divided form.

The compositions containing a compound of this invention may also comprise an additional agent selected from the group consisting of cortiocosteroids, bronchodilators, antiasthmatics (mast cell stabilizers), anti-inflammatories, antirheumatics, immunosuppressants, antimetabolites, immunomodulators, antipsoriatics, and antidiabetics. Specific compounds include theophylline, sulfasalazine and aminosalicylates (anti-inflammatories); cyclosporin, FK-506, and rapamycin (immunosuppressants); cyclophosphamide and methotrexate (antimetabolites); and interferons (immunomodulators).

An aerosol composition suitable for use as the inhalable form may comprise the compounds of the invention in solution or dispersion in a propellant, which may be chosen from any of the propellants known in the art. Suitable such propellants include hydrocarbons such as n-propane, n-butane or isobutane or mixtures of two or more such hydrocarbons, and halogen-substituted hydrocarbons, for example fluorine-substituted methanes, ethanes, propanes, butanes, cyclopropanes or cyclobutanes, particularly 1,1,1,2-tetrafluoroethane (HFA134a) and heptafluoropropane (HFA227), or mixtures of two or more such halogen-substituted hydrocarbons. Where the compounds of the invention are present in dispersion in the propellant, i.e. where present in particulate form dispersed in the propellant, the aerosol composition may also contain a lubricant and a surfactant, which may be chosen from those lubricants and surfactants known in the art. The aerosol composition may contain up to about 5% by weight, for example 0.002 to 5%, 0.01 to 3%, 0.015 to 2%, 0.1 to 2%, 0.5 to 2% or 0.5 to 1%, by weight of the compounds of the invention, based on the weight of the propellant. Where present, the lubricant and surfactant may be in an amount up to 5% and 0.5% respectively by weight of the aerosol composition. The aerosol composition may also contain ethanol as co-solvent in an amount up to 30% by weight of the composition, particularly for administration from a pressurized metered dose inhalation device.

A finely divided particulate form, i.e. a dry powder, suitable for use as the inhalable form may comprise the compounds of the invention in finely divided particulate form, optionally together with a finely divided particulate carrier, which may be chosen from materials known as carriers in dry powder inhalation compositions, for example saccharides, including monosaccharides, disaccharides and polysaccharides such as arabinose, glucose, fructose, ribose, mannose, sucrose, lactose, maltose, starches or dextran. As especially preferred carrier is lactose. The dry powder may be in capsules of gelatin or plastic, or in blisters, for use in a dry powder inhalation device, preferably in dosage units of 5 .mu.g to 40 mg of the active ingredient. Alternatively, the dry powder may be contained as a reservoir in a multi-dose dry powder inhalation device.

In the finely divided particulate form, and in the aerosol composition where the compounds of the invention are present in particulate form, the compound of the invention may have an average particle diameter of up to about 10 .mu.m, for example 1 to 5 .mu.m. The particle size of the compound of the invention, and that of a solid carrier where present in dry powder compositions, can be reduced to the desired level by conventional methods, for example by grinding in an air-jet mill, ball mill or vibrator mill, microprecipitation, spray-drying, lyophilisation or recrystallisation from supercritical media.

The inhalable medicament comprising the pharmaceutical compositions of the invention may be administered using an inhalation device suitable for the inhalable form, such devices being well known in the art. Accordingly, the invention also provides a pharmaceutical product comprising the compounds of the invention in inhalable form as hereinbefore described in association with an inhalation device. In a further aspect, the invention provides an inhalation device containing the compounds of the invention in inhalable form as hereinbefore described.

Where the inhalable form is an aerosol composition, the inhalation device may be an aerosol vial provided with a valve adapted to deliver a metered dose, such as 10 to 100 .:1, e.g. 25 to 50 .:1, of the composition, i.e. a device known as a metered dose inhaler.

Suitable such aerosol vials and procedures for containing within them aerosol compositions under pressure are well known to those skilled in the art of inhalation therapy. Where the inhalable form is a nebulizable aqueous, organic or aqueous/organic dispersion, the inhalation device may be a known nebulizer, for example a conventional pneumatic nebulizer such as an aij et nebulizer, or an ultrasonic nebulizer, which may contain, for example, from 1 to 50 mL, commonly 1 to 10 mL, of the dispersion; or a hand-held nebulizer such as an AERX (ex Aradigm, US) or BINEB (Boehringer Ingelheim) nebulizer which allows much smaller nebulized volumes, e.g. 10 to 100 .mu.1, than conventional nebulizers. Where the inhalable form is the finely divided particulate form, the inhalation device may be, for example, a dry powder inhalation device adapted to deliver dry powder from a capsule or blister containing a dosage unit of the dry powder or a multidose dry powder inhalation device adapted to deliver, for example, 25 mg of dry powder per actuation. Suitable such dry powder inhalation devices are well known.

The pharmaceutical compostions of the invention may be synthesized using known techniques. According to one embodiment, the naphthalimides used in the present invention is amonafide synthesized according to a method disclosed in U.S. provisional application Ser. No. 60/394,558, filed Jul. 8, 2002, hereby incorporated by reference in its entirety.

Kits

The present invention provides kits suitable for treatment of a host with a cellular proliferative disease. In one embodiment, the kit includes a first container comprising a composition comprising a naphthalamide. In one aspect, the naphthalimide is amonafide. In another embodiment, the kit includes a second container comprising a compositions comprising an antimetabolite. In one aspect, the antimetabolite is cytarabine. In another embodiment, the kit comprises a one container comprising a composition comprising a naphthalimide and an antimetabolite.

In one other embodiment, the compositions contained in the kit as described herein are lyophilized. In another embodiment, the kit further comprises a third container comprising a solvent for reconstitution of the lypophilized compositions. The solvent may be DMSO, saline, water or any other suitable media for reconstitution. In one other embodiment, the kit includes a needle for injection of the compositions as described herein into a host with a cellular proliferative disease.

The present invention provides a kit as described herein with instructions to treat a host with a cellular proliferative disease. The kits of the present invention may further include instructions for use. Instructions may be included as a separate insert and/or as part of the packaging or container, such as a label affixed to a container or as a writing or other communication integrated as part of a container. The instructions may inform the user of methods of administration of the compositions contained therein, precautions, expected results, warnings concerning improper use, and the like.

In one embodiment, the instructions provide directions on how to reconstitute the compositions described herein. In another embodiment, the instructions provide directions on how to prepare the compositions of the invention for administration. In yet another embodiment, the instructions provide directions to administer naphthalimide at a dosage of about 0.5 mg/kg to about 125 mg/kg, about 1 mg/kg to about 120 mg/kg, about 2 mg/kg to about 115 mg/kg, about 5 mg/kg to about 110 mg/kg, about 10 mg/kg to about 105 mg/kg, about 15 mg/kg to about 100 mg/kg, about 20 mg/kg to about 100 mg/kg, about 25 mg/kg to about 95 mg/kg, about 30 mg/kg to about 90 mg/kg, about 35 mg/kg to about 85 mg/kg, about 40 mg/kg to about 80 mg/kg, about 45 mg/kg to about 75 mg/kg, about 50 mg/kg to about 70 mg/kg, about 55 mg/kg to about 65 mg/kg, about 57 mg/kg to about 63 mg/kg, and preferably about 60 mg/kg. In one aspect, the naphthalimide is amonafide.

In one another embodiment, the instructions provide directions to administer a metabolite at a dosage of between about 1 mg/kg to about 800 mg/kg, about 25 mg/kg to about 775 mg/kg, about 50 mg/kg to about 750 mg/kg, about 75 mg/kg to about 725 mg/kg, about 100 mg/kg to about 700 mg/kg, about 125 mg/kg to about 675 mg/kg, about 150 mg/kg to about 650 mg/kg, about 175 mg/kg to about 625 mg/kg, about 200 mg/kg to about 600 mg/kg, about 225 mg/kg to about 575 mg/kg, about 250 mg/kg to about 550 mg/kg, about 275 mg/kg to about 525 mg/kg, about 300 mg/kg to about 500 mg/kg, about 325 mg/kg to about 475 mg/kg, about 350 mg/kg to about 450 mg/kg, about 375 mg/kg to about 425 mg/kg, and preferably about 400 mg/kg. In one aspect the metabolite is cytarabine.

The kits as described herein may further comprise suitable packaging of the respective compositions, instructions, and/or other optional components as disclosed below. In one embodiment, kits of the present invention may further contain components useful in the application of the compositions described herein. Other components include without limitation chemical-resistant disposal bags, applicators, bodily surface-cleansing agents such as alcohol swabs, diluent, towels or towellettes, gloves, scissors, marking pens and eye protection.

The following examples are offered by way of illustration and not by way of limitation.

### EXAMPLE 1

### Chemopotentiation of Cisplatin by Amonafide

Transplantable experimental murine fibrosarcomas (2×105 RIF-1 cells) were grown intradermally in the flanks of 3 month old female C3H mice (Charles River, Holister, Calif.). When the tumors reached a volume of approximately 100 mm3, the mice were randomly assigned to each experimental group (4 mice per group).

The experimental compositions were prepared as described in Table 2.

TABLE 2

| Agent | Dose | Solvent | Supplier |
|---|---|---|---|
| Amonafide | 50 mg/kg | DMSO | NCI |
| Cisplatin | 4 mg/kg | Water for injection | David Bull Labs |

The chemopotentiator, amonafide, was obtained from NCI and was made to the appropriate concentration in DMSO. Cisplatin (David Bull Laboratories-Mulgrave, Australia, lot. 5201844x) was made to the appropriate concentration in water for injection. The compositions were injected systemically (i.e., intraperitoneally, i.p.), in a volume of 100 microliters. For the treatment of group 3, the chemopotentiator, amonafide, was injected 30 minutes prior to the injection of cisplatin. After treatment, the growth of the tumors was monitored three times per week by caliper measurements of three perpendicular diameters of the tumor and calculation of tumor volume from the formula:
V=π/6×D₁×D₂×D₃,
where D₁₋₃ represents tumor diameters, in mm.

The tumors were followed until they reached a size of four times their day zero treatment volume (TVQT), or up to 30 days after treatment, whichever came first. The data is expressed as the “tumor volume quadrupling time” (TVQT) mean and as the “delay.” Mean TVQT is the mean days required for individual tumors to grow to four times the tumor volume at the initial treatment day. The “delay” is the median of days required for a tumor to grow to four times the mean size of the treated group, minus the median of days required to grow to four times the mean size of the control group. The data is also expressed as the ratio of the tumor volume quadrupling time of the treated tumor over the untreated control group (TVQT/CTVQT). Increasing values of this ratio indicate increased antitumor response.

The data is presented in Table 3 below and in FIG. 2.

TABLE 3

| | | Dose | Mean | TVQT/ | Median | Delay |
|---|---|---|---|---|---|---|
| Group | Treatment | (mg/kg) | TVQT ± S.E. | CTVQT | (TVQT) | (Days) |
| 1 | Untreated Control | — | 6.3 ± 0.3 | 1.0 | 6 | 0.00 |
| 2 | Amonafide | 50 | 9.7 ± 0.6 | 1.5 | 9.0 | 2.94 |
| 3 | Amonafide → Cisplatin | 50 → 4 | 17.9 | 2.8 | 17.9 | 11.81 |
| 4 | Cisplatin | 4 | 8.4 ± 0.3 | 1.3 | 8.1 | 2.10 |

The arrow → in Group 3 indicates administration 30 minutes following administration of amonafide.

The results of Table 3 indicate that the antiproliferative activity of cisplatin is enhanced by the use of the chemopotentiator, amonafide in that a more than additive effect was observed when both compounds were used to treat the tumor bearing mice (group 3) in comparison to the use of cisplatin alone (group 4) or amonafide alone (group 2).

### EXAMPLE 2

### Effect of Amonafide, Alone and in Combination with Other Chemotherapeutics on RIF-1 Tumor Growth in C3H Mice

The RIF-1 murine fibrosarcoma tumor model was used to evaluate the antitumor activity of amonafide, alone and and in combination with various antiproliferative agents. The antiproliferative agents used include those that affect nucleic acid (e.g., DNA) integrity (e.g., cisplatin, etoposide, 5-fluorouracil), agents that affect structural or cytoplasmic proteins or their synthesis (e.g., homoharringtonine, paclitaxel, vinblastine, colchicine, curcumin or parthenolide).

Amonafide-NCI was obtained from NCI as a powder. Amonafide-Penta was obtained from Penta Biotech (Union City, Calif.), Lot No. 039-01, as a powder. Cisplatin for Injection, USP, was obtained from David Bull Labs (Mulgrave, Australia), Lot No. 5201844x, as a lypholized powder. Paclitaxel was obtained from Bristol Myers Squibb Co. (Princeton, N.J.), Lot No. 9J16241, exp. September 2001, prediluted to 6 mg/mL in Cremaphor/EL. Vinblastine was obtained from Bedford Labs (Bedford, Ohio), Lot No. 112647, as a lypholized powder. Etoposide was obtained from Pharmacia (Kalamazoo, Mich.), Lot No. ETA013, exp. 5/99, as a liquid prediluted to 20 mg/mL. 5-Fluorouracil was obtained from Pharmacia (Kalamazoo, Mich.), Lot No. FFA191, exp. 7/00, as a liquid prediluted to 50 mg/mL. Curcumin was obtained from Sigma (St. Louis, Mo.), Lot No. 69H3457. Parthenolide was obtained from Tocris (Ballwin, Mo.) Lot. No. 7/18089. DMSO was obtained from Sigma (St. Louis, Mo.), Lot No. 80K3695. 0.9% Sodium Chloride for Injection, USP (saline) was manufactured by Abbott Laboratories (Lot No. 55-199-DK). Sterile Water for Injection, USP (WFI) was manufactured by Lyphomed, Inc. (Lot No. 390849).

Formulations: Test preparations (treatment groups) are summarized in Table 4.

TABLE 4
Summary of Treatment Groups

| | | Concen- | Route of | Injection |
|---|---|---|---|---|
| Formu- | | tration | Admini- | Volume |
| lation | Treatment | (mg/mL) | stration | (μL) |
| 1 | Amonafide-NCI in DMSO | 12.5 | IP | 100 |
| 2 | Amonafide-Penta in DMSO | 12.5 | IP | 100 |
| 3 | Amonafide-Penta in Saline | 7.5 | IP | 100 |
| 4 | CDDP in WFI | 1 | IP | 100 |
| 5 | Paclitaxel in WFI | 2.5 | IP | 100 |
| 6 | Vinblastine in saline | 0.5 | IP | 100 |
| 7 | Etoposide in saline | 2.5 | IP | 100 |
| 8 | 5-Fluorouracil in saline | 3.75 | IP | 100 |
| 9 | 5-Fluorouracil in saline | 7.5 | IP | 100 |
| 10 | Colchicine in saline | 2.5 | PO | 100 |
| 11 | HHT-Clin in WFI | 1 | IP | 100 |
| 12 | Curcumin in DMSO | 6.25 | IP | 100 |
| 13 | Parthenolide in DMSO | 5 | IP | 100 |

For preparation of formulation 1 and 2, amonafide was weighed into vials and dissolved in DMSO at 12.5 mg/mL.

For formulation 3, amonafide was weighed into vials and dissolved in saline.

For formulation 4, the contents of a 10-mg vial of lyophilized CDDP (Cisplatin for Injection) was resuspended with 10 mL WFI to produce a 1 mg/mL CDDP suspension.

For formulation 5, paclitaxel, prediluted in Cremaphor/EL and dehydrated alcohol to 6 mg/mL was further diluted to 3.3 mg/mL with WFI.

Formulation 6 was made by adding 0.9% Sodium Chloride for Injection to a vial of 10 mg of vinblastine lypholized powder.

Formulations 7-10 were prepared by diluting the appropriate amount of each test agent into saline (7-2.5 mg/mL etoposide, 8-7.5 mg/mL 5-fluorouracil, 9-3.75 mg/mL 5-fluorouracil 10-2.5 mg/mL colchicine,).

Formulation 11 was undiluted HHT-Clin, used as received.

Formulations 12 and 13 were prepared by diluting the appropriate amount of each test agent into DMSO (12-6.25 mg/mL curcumin and 13-5 mg/mL parthenolide).

Animals: Female C3H mice (Charles River Laboratories, Holister, Calif.), approximately 3 months old, were used for the study. The average body weight was approximately 25 g. Animals were maintained in isolator cages on a 12-hour light-and-dark cycle. Food and water were available ad libitum.

Tumors: The RIF-1 murine fibrosarcoma cell line was maintained in in vitro culture (Waymouth medium supplemented with 20% fetal bovine serum) at 37 C. in a humidified 5% CO2 incubator. Log-phase RIF-1 cells were trypsinized and harvested from cell culture flasks to yield a concentration of 4×10⁶ cells/mL, then injected intradermally in a volume of 50 μL (equivalent to 2×10⁵ cells per injection) into both flanks of each mouse. Nine days later, when tumors reached approximately 100 mm³ in size, the animals were randomized to different treatment groups.

Treatment Groups: Treatment groups are summarized in Table 4. Four to five animals were assigned to each treatment group. The intraperitoneal injection volume was 100 μL. The oral administration volume was 100 μL. Combination treatments using two test agents were administered as two separate injections, with the second one following the first either immediately or after 30 minutes.

Evaluation of Tumor Growth Delay: Tumors were measured three times weekly for up to 22 days with Vernier calipers. Tumor volume (cubic millimeters, mm³) was calculated according to the formula: V=π/6×D1×D2×D3 in which D1-3 are perpendicular diameters measured in millimeters (mm).

Tumor volume quadrupling time (TVQT), defined as the time required for a tumor to grow to four times (4×) its initial volume (at the time of treatment), was used as a study endpoint. The TVQT was determined for each treatment group and expressed in days as the mean±standard error (SE).

Antitumor activity or modulation of tumor growth (as measured by delayed tumor growth, i.e. increases in TVQT values) by amonafide administered as a single agent or in combination with other chemotherapeutics is presented in Table 5.

TABLE 5
Effect of Amonafide and Amonafide in Combination with Other
Chemotherapeutics on RIF-1 Tumor Growth in C3H Mice

| | | Drug Dose | Route of | Number of | |
|---|---|---|---|---|---|
| Group | Treatment | (mg/Kg) | Administration | Tumors | TVQT |
| 1 | Untreated Control | — | — | 40 | 7.0 ± 0.2 |
| 2 | Amonafide-NCI/DMSO | 50 | IP | 8 | 9.7 ± 0.6 |
| 3 | Amonafide-Penta/DMSO | 50 | IP | 8 | 9.3 ± 0.3 |
| 4 | Amonafide-Penta/Saline | 30 | IP | 12 | 7.3 ± 0.2 |
| 5 | Cisplatin/WFI | 4 | IP | 16 | 9.2 ± 0.4 |
| 6 | Paclitaxel/Cremaphor EL | 10 | IP | 8 | 7.9 ± 0.3 |
| 7 | Vinblastine/Saline | 2 | IP | 8 | 8.6 ± 0.4 |
| 8 | Etoposide/Saline | 10 | IP | 8 | 8.5 ± 0.5 |
| 9 | Fluorouracil/Saline | 15 | IP | 8 | 6.7 ± 0.4 |
| 10 | Fluorouracil/Saline | 30 | IP | 8 | 13.6 ± 1.9 |
| 11 | Homoharringtonine/WFI | 4 | IP | 8 | 8.5 ± 0.5 |
| 11 | Colchicine/Saline | 10 | PO | 8 | 6.3 ± 0.3 |
| 12 | Curcumin/DMSO | 25 | IP | 8 | 9.7 ± 1.1 |
| 13 | Parthenolide/DMSO | 20 | IP | 8 | 8.5 ± 0.8 |
| 14 | Amonafide-NCI/DMSO-30 CDDP/WFI | 50, 4 | IP, IP | 4 | 17.9 ± 0.4 |
| 15 | Amonafide-Penta/Saline-10 sec- | 30, 4 | IP, IP | 8 | 11.0 ± 0.4 |
| | CDDP/WFI | | | | |
| 16 | Amonafide-Penta/DMSO-10 sec- | 30/10 | IP, IP | 8 | 9.8 ± 0.4 |
| | Paclitaxel/Cremaphor EL | | | | |
| 17 | Amonafide-Penta/Saline- | 30, 2 | IP, IP | 8 | 9.5 ± 1.1 |
| | 10 sec-Vinblastine/Saline | | | | |
| 18 | Amonafide-Penta/Saline- | 30, 10 | IP, IP | 8 | 8.5 ± 0.9 |
| | 10 sec-Etoposide/Saline | | | | |
| 19 | Amonafide-Penta/Saline-10 sec- | 30, 15 | IP, IP | 8 | 7.7 ± 0.8 |
| | 5-Fluorouracil/Saline | | | | |
| 20 | Amonafide-Penta/Saline-10 sec- | 30, 30 | IP, IP | 8 | 20.2 ± 1.0 |
| | 5-Fluorouracil/Saline | | | | |
| 21 | Amonafide/WFI-10 sec- HHT-Clin/WFI | 30, 4 | IP, IP | 8 | 10.2 ± 0.5 |
| 22 | Amonafide-Penta/Saline-10 sec- | 30, 10 | IP, PO | 8 | 7.1 ± 0.3 |
| | Colchicine/WFI | | | | |
| 23 | Amonafide-Penta/Saline-10 sec- | 30/25 | IP, IP | 8 | 8.2 ± 0.2 |
| | Curcumin | | | | |
| 24 | Amonafide-Penta/Saline-10 sec- | 30/20 | IP, IP | 8 | 7.6 ± 0.3 |
| | Parthenolide | | | | |

Results from five separate experiments are included in this study. Untreated control animals quadrupled in size in an average of 7.0 days. Intraperitoneal administration of amonafide-NCI formulated in DMSO at 50 mg/Kg had a TVQT of 9.7 days. The additional intraperitoneal administration of CDDP further extended the mean TVQT to 17.9 days. Intraperitoneal administration of amonafide-Penta formulated in DMSO at 50 mg/Kg had a TVQT of 9.3 days. While paclitaxel (10 mg/Kg), alone, demonstrated a TVQT of 7.9 days, the addition of amonafide (50 mg/kg) extended the TVQT to 9.8 days.

Amonafide-Penta formulated in saline at 30 mg/Kg was used for the remainder of the combination studies.

At 30 mg/Kg, amonafide had an average TVQT of 7.3 days. Combination administration of cisplatin (4 mg/Kg) with amonafide (30 mg/Kg) yielded a TVQT of 11.0 days, which was greater than amonafide (TVQT=7.3 days) or cisplatin (TVQT=9.2 days), alone.

Administration of amonafide (30 mg/Kg) in combination with 5-fluorouracil (30 mg/Kg) resulted in a TVQT of 20.2 days versus 13.6 days for 5-fluorouracil, alone. At a dose of 15 mg/Kg, 5-fluorouracil gave a TVQT of 6.7 days versus 7.7 days when it was combined with amonafide at 30 mg/Kg. Combination administration of amonafide (30 mg/Kg) and vinblastine (2 mg/Kg) yielded a TVQT of 9.5 days versus 8.6 days for vinblastine, alone. Combination administration of amonafide (30 mg/Kg) and homoharringtonine (4 mg/Kg) yielded a TVQT of 10.2 days, versus 8.5 for homoharringtonie, alone. Amonafide in combination with etoposide(10 mg/Kg) gave a TVQT of 8.5 days which was the same as the TVQT for etoposide, alone. Combinations of amonafide with curcumin or parthenolide yielded TVQT's of 8.2 days and 7.6 days, respectively, which was less than curcumin (TVQT=9.7 days) or parthenolide (TVQT=8.5) as individual agents.

Orally administered colchicine (10 mg/Kg) yielded a TVQT of 6.3 days. Amonafide in combination with colchicine increased the TVQT to 7.1 days.

There were amnmal deaths in some groups that were recorded as follows: Two of four mice died after treatment of amonafide-NCI formulated in DMSO at 12.5 mg/mL.

In summary, intraperitoneal administration of amonafide had antitumor activity in the RIF-1 murine fibrosarcoma tumor model. Intraperitoneal administration of amonafide in combination with cisplatin, paclitaxel, vinblastine, 5-fluorouracil and homoharringtonine had antitumor activity levels greater than amonafide alone, or the individual test agents. The best combinatorial activities used cisplatin, 5-fluorouracil, and homharringtonine. Amonafide in combination with colchicine had antitumor activity less than amonafide alone. Amonafide in combination with etoposide, curcumin or parthenolide was greater than that of amonafide alone, but less than that of the test agents individually.

### EXAMPLE 3

### Effect of Amonafide in Combination with Camptothecin, Genistein or Rosmarinic Acid on RIF-1 Tumor Growth in C3H Mice.

Transplantable experimental murine fibrosarcomas (2×10⁵ RIF-1 cells) were grown intradermally in the flanks of 3 month old female C3H mice (Charles River, Holister, Calif.). When the tumors reached a volume of −100 mm³, the mice were randomly assigned to each experimental group (4 mice per group).

The experimental compositions were prepared as described in Table 6.

TABLE 6

| Agent | Dose | | Solvent | Supplier |
|---|---|---|---|---|
| Amonafide | 30 | mg/Kg | saline | Penta |
| Camptothecin | 6 | mg/Kg | DMSO | Boehinger Ingelheim |
| Genistein | 60 | mg/Kg | DMSO | ChemCon GmbH |
| Rosmarinic Acid | 20 | | DMSO | Tocris |

Amonafide was manufactured by Penta for ChemGenex and was made to the appropriate concentration in saline. Genistein (ChemCon GmbH, Lot CC6700-26) Rosmarinic acid (Tocris- Batch 2/18077) and Camptotheicn (Boehinger Ingelheim- Lot 142088) were made to the appropriate concentrations in DMSO. The compositions were injected systemically (i.e., intraperitoneally, i.p.), in a volume of 100 μl. For the treatment of groups 3, 5 and 7, amonafide, was injected immediately prior to the injection of camptothecin, genistein or rosmarinic acid. After treatment, the growth of the tumors was monitored three times per week by caliper measurements of three perpendicular diameters of the tumor and calculation of tumor volume from the formula:
V=π/6×D₁×D₂×D₃,
where D₁₋₃ represents tumor diameters, in mm.

The data is also expressed as the tumor growth delay (TGD) median which is the median days to 4× the tumor volume from the initial treatment day, and as the delay which is the median days to 4× of the treated group minus the median days to 4× of the control group. The T/C ratio is the ratio of days to 4× of the treated tumors over the days to 4× of the untreated control tumors. Increasing values indicate increased antitumor response.

The tumors were followed until they reached 4 times their Day 0 treatment volume or up to 30 days after treatment (tumor growth delay, TGD), whichever came first. The data is also expressed as the ratio of the tumor growth delay of the treated tumor (TGD) over the untreated control group (CTGD). Increasing values of this ratio indicate increased antitumor response.

The data are presented in Table 7.

TABLE 7

| | | Dose | | TGD/ | Median | Delay |
|---|---|---|---|---|---|---|
| Group | Treatment | (mg/kg) | TGD ± S.E. | CTGD | (TGD) | (Days) |
| 1 | Untreated Control | — | 7.5 ± 0.6 | 0.0 | 7.3 | 0.00 |
| 2 | Amonafide | 30 | 7.0 ± 0.4 | 0.9 | 7.0 | −0.36 |
| 3 | Amonafide + Camptothecin | 30/6 | 14.9 ± 0.4 | 2.0 | 14.8 | 7.47 |
| 4 | Camptothecin | 6 | 12.9 ± 0.5 | 1.7 | 12.8 | 5.45 |
| 5 | Amonafide + Genistein | 30/60 | 8.6 ± 0.2 | 1.1 | 8.6 | 1.27 |
| 6 | Genistein | 60 | 7.9 ± 0.4 | 1.1 | 8.1 | 0.78 |
| 7 | Amonafide + Rosmarinic Acid | 30/20 | 8.9 ± 0.5 | 1.2 | 8.5 | 1.18 |
| 8 | Rosmarinic Acid | 20 | 8.4 ± 0.5 | 1.1 | 7.8 | 0.46 |

The results of Table 7 indicate that the antiproliferative activity of camptothecin, genistein and rosmarinic acid was enhanced by the use of the chemopotentiator, amonafide in that a more than additive effect was observed when both compounds were used to treat the tumor bearing mice (groups 3, 5 and 7) in comparison to the use of camptothecin, genistein or rosmarinic acid alone (groups 4, 6 and 8) or amonafide alone (group 2).

### EXAMPLE 4

### Chemopotentiation of Cytarabine by Amonafide

Transplantable experimental murine fibrosarcomas (2×10⁵ RIF-1 cells) were grown intradermally in the flanks of 3 month old female C3H mice (Harlan, Indianapolis, Ind.). When the tumors reached a volume of approximately 100 mm³, the mice were randomly assigned to each experimental group (4 mice per group).

The experimental compositions were prepared as described in Table 8.

TABLE 8

| Agent | Dose | Solvent | Supplier |
|---|---|---|---|
| Amonafide | 60 mg/kg | Saline | ChemGenex |
| dihydrochloride | | | Pharmaceuticals, Inc. |
| Cytarabine | 400 mg/kg | Water for | Bedford Labs |
| | | injection | |

The chemopotentiator, amonafide, was obtained from ChemGenex Pharmaceuticals, Inc. and was made to the appropriate concentration in saline. Cytarabine (Bedford Laboratories—Bedford, Ohio) was made to the appropriate concentration in water for injection. The compositions were injected systemically (i.e., intraperitoneally, i.p.), in a volume of 100 microliters. After treatment, the growth of the tumors was monitored three times per week by caliper measurements of three perpendicular diameters of the tumor and calculation of tumor volume from the formula:
V=π/6×D₁×D₂×D₃,
where D₁₋₃ is in mm.

The tumors were followed until they reached a size of four times their day zero treatment volume (TVQT), or up to 30 days after treatment, whichever came first. The data is expressed as the “tumor volume quadrupling time” (TVQT) mean and as the “delay.” Mean TVQT is the mean days required for individual tumors to grow to four times the tumor volume at the initial treatment day. The “delay” is the median of days required for a tumor to grow to four times the mean size of the treated group, minus the median of days required to grow to four times the mean size of the control group. The data is also expressed as the ratio of the tumor volume quadrupling time of the treated tumor over the untreated control group (TVQT/CTVQT). Increasing values of this ratio indicate increased antitumor response.

The data is presented in Table 9 below and in FIG. 2.

TABLE 9

| | | | Mean | | | |
|---|---|---|---|---|---|---|
| | | Dose | TVQT ± | TVQT/ | Median | Delay |
| Group | Treatment | (mg/kg) | S.E. | CTVQT | (TVQT) | (Days) |
| 1 | Untreated | — | 5.9 ± 0.7 | 1.0 | 5.5 | 0.00 |
| | control | | | | | |
| 2 | Amonafide | 60 | 8.7 ± 1.1 | 1.5 | 7.4 | 1.88 |
| 3 | Cytarabine | 400 | 7.0 ± 0.8 | 1.2 | 6.5 | 1.04 |
| 4 | Cytarabine + | 60 | 9.8 ± 0.7 | 1.7 | 10.2 | 4.73 |
| | Amonafide | 400 | | | | |

For group 4, amonafide was administered immediately after cytarabine.

The results of Table 9 indicate that the antiproliferative activity of cytarabine is enhanced by the use of the chemopotentiator, amonafide in that a more than additive effect was observed when both compounds were used to treat the tumor bearing mice (group 4) in comparison to the use of cytarabne alone (group 3) or amonafide alone (group 2).

We claim:

1. A method of treatment of a host with a cellular proliferative disease, comprising contacting said host with a naphthalimide in conjunction with cytarabine, each in an amount sufficient to modulate said cellular proliferative disease.

2. The method of claim 1 wherein said cellular proliferative disease is a solid tumor.

3. The method according to claim 1, wherein said naphthalimide comprises amonafide.

4. The method according to claim 1, wherein said naphthalimide comprises an amonafide analog.

5. The method according to claim 1 wherein said naphthalimide has a chemopotentiating effect.

6. The method according to claim 5 wherein said naphthalimide is amonafide.

7. The method according to claim 1 wherein said host is a human.

8. The method according to claim 1 wherein said naphthalimide is administered before the administration of said cytarabine.

9. The method according to claim 1 wherein said naphthalimide is administered during the administration of said cytarabine.

10. The method according to claim 1 wherein said naphthalimide is administered after the administration of said cytarabine.

11. The method according to claim 10, wherein said naphthalimide is administered less than 4 hours after the administration of said cytarabine.

12. The method according to claim 1, wherein said naphthalimide is administered less than 4 hours before the administration of said cytarabine.

13. The method of claim 1 wherein the modulation of said disease with said composition is greater than that for said cytarabine alone.

14. The method of claim 2 wherein said contacting results in an increase in tumor volume quadrupling time for said solid tumor.

15. The method of claim 1 wherein said contacting step comprises at least one intraperitoneal injection.

16. A method of treatment of a host with a cellular proliferative disease comprising contacting said host with a naphthalimide in conjunction with cytarabine, each in an amount sufficient to modulate said cellular proliferative disease.

17. The method of claim 16 wherein said cellular proliferative disease is a solid tumor.

18. The method of claim 16 wherein said naphthalimide is amonafide.

19. A composition comprising a naphthalimide and cytarabine.

20. The composition of claim 19 wherein said naphthalimide is amonafide.

21. A pharmaceutical formulation comprising naphthalimide, an cytarabine, and a pharmaceutically acceptable excipient.

22. The pharmaceutical formulation of claim 21 wherein said naphthalimide is amonafide.

23. The pharmaceutical formulation of claim 21 wherein said formulation is suitable for intraperitoneal injection.

24. A kit for the treatment of a host with a cellular proliferative disease, comprising a naphthalimide and cytarabine, each in an amount sufficient to modulate said cellular proliferative disease.

25. The kit of claim 24 wherein said kit further comprises instructions for treating a cellular proliferative disease.

26. The kit of claim 24 wherein said naphthalimide is amonafide.
